(19) United States
(12) Patent Application Publication
(10) Pub. No.: US 2009/0311324 A1
(43) Pub. Date: Dec. 17, 2009

(54) POLYMER MATRIX, METHOD FOR ITS PRODUCTION, AND ITS USE

(75) Inventors: Ute Steinfeld, St. Ingbert (DE); Barbara Palm, Kirkel-Limbach (DE); Hyeck Hee Lee, St. Ingbert (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: KIST-Europe Forschungsgesellschaft mbH, Saarbrucken (DE)

(21) Appl. No.: 12/439,449

(22) PCT Filed: Aug. 31, 2007
(86) PCT No.: PCT/EP2007/007632

§ 371 Date: Aug. 11, 2009

(30) Foreign Application Priority Data
Aug. 31, 2006 (DE) 10 2006 040 772.5

(51) Int. Cl. A61K 9/00; C08F 20/06; C08F 20/10; C08F 20/56; C08G 79/02; C08G 63/00; C08F 26/10; C08G 63/06; C08G 18/00; C08G 77/04; C08G 65/04; C08F 16/06; C08F 10/02; C08B 37/00; C08B 37/08; A61P 43/00; C12N 5/00; C12N 13/00; A61K 47/30; A61K 47/36

(52) U.S. Cl. 424/484; 526/317.1; 526/320; 526/303.1; 528/399; 528/271; 526/258; 528/361; 528/44; 528/10; 528/421; 525/56; 526/352; 536/102; 536/56; 536/20; 525/54.1; 525/54.2; 435/375; 435/173.9; 514/772.3; 514/777

(57) ABSTRACT

The present invention relates to a polymer matrix for specifically tethering surface epitopes or receptors of cells, said polymer matrix having been pretreated by molecular imprinting. The invention also relates to a method of producing such a polymer matrix. The polymer matrices according to the present invention are used in therapeutic and diagnostic applications as well as for isolating cells.

Currently, drugs are specifically delivered to the body via a large number of systems that are characterized in that they are able to hold a large amount of drug per particle and that they have a biocompatible shell and therefore have a low toxicity. In addition, it is possible to adjust the release rate and the site of action. To this end, there are many different types of particles, e.g., micelles, within which the drug can be enclosed. The shell can be chemically modified so that a premature degradation in the blood stream is avoided.

In addition, liposomes, dendrimers, hydrogels and particles with the advantages mentioned above also serve to deliver drugs, with the drug, depending on the type of particles used, being contained in the core or being uniformly distributed over the entire particle. Through the structure of the shell or the attachment of specific binding sites, the shell can be modified in such a manner that, e.g., cancer cells can be specifically targeted by the particles, in which cells the drug is subsequently released, either by dissolving the particle or due to secondary chemical or physicochemical reactions. Examples to be mentioned are changes in the pH value or in the temperature. The particles can also be taken up by the target cells where they are subsequently dissolved, e.g., as a result of the conditions prevailing in the cell, by enzymatic processes. Because of the versatile possibilities of structuring the particles, the particles can be tailor-made to meet the specific requirements of the system to be treated.

A more recent mechanism of delivering drugs uses molecularly imprinted polymers (MIP). In this case, functional and crosslinking polymers are polymerized to form nanoparticles in the presence of the drug to be delivered. In this situation, the drug serves as a template which, in a subsequent step, is washed out of the polymers produced and which leaves cavities that have the specific configuration and size for the template and therefore can react specifically to it, with the degree of specificity desired being choosable. Thus, the advantages of MIPs are high affinity and specificity for the target molecules which correspond to those of natural receptors, their stability which is higher than that of their biological representatives, and their simple synthesis and adaptability to the conditions that have to be met in a given application.

It is also possible to produce the polymer in the presence of the enantiomer or a diastereomer of the drug (in the case of chiral active substances) or even structural analogs. In this case, the active substance itself has a slightly lower affinity to the polymer, which facilitates the release of the active substance. In another possible embodiment, the polymer is imprinted with a substance which is present at the target site, which structure has structural similarities with the active substance; as a result, the active substance is again bonded less strongly in the polymer and is displaced at the target site by the imprinting substance. The drug can be released by diffusion, displacement, (biological) degradation of the substrate material, or a combination of the above.

Existing MIP systems are often selected to ensure that in addition to the drug to be delivered, the systems also carry receptors or antibodies for recognizing specific epitopes on the target cell where they release their cargo. They circulate through the bloodstream until they are gradually taken up by the target cells. Thus, the bloodstream serves as the direct means of delivery. Such systems are known from the following published documents:

O. Kotrotsiou, K. Kotti, E. Dini, O. Kammona and C. Kiparissides, Journal of Physics: Conference Series 10 (2005), pp. 281-284,

S. Alexandridou and C. Kiparissides, Proc. of the EC-NSF Workshop on Nanotechnology—Revolutionary Opportunities and Societal Implications (Lecce, Italy, Jan. 31-Feb. 1, 2002),

M. E. Byrne, K. Park and N. A. Peppas, Adv. Drug Delivery Rev. 54 (2002), pp. 149-161, and

N. V. Majeti Ravi Kumar, J. Pharm. Pharmaceut. Sci. 3 (2) (2000), pp. 234-258.

Using these systems as a starting point, the problem to be solved by the present invention was to make available a simple method by means of which surface epitopes or receptors of cells can be selectively recognized or tethered.

This problem is solved with the polymer matrix with the characteristics of claim 1 and with the method with the characteristics of claim 18. In claims 24-27, applications according to the present invention are enumerated. Useful improvements follow from the other dependent claims.

According to the present invention, a polymer matrix for specifically tethering surface epitopes or receptors of cells is made available, with the polymer matrix having molecularly imprinted surface epitopes or receptors and binding cavities specific for these surface epitopes or receptors.

The present invention makes it possible to use the molecular imprinting method to produce polymer matrices, in particular polymer particles, on which receptors, antibodies or bispecific antibodies are replaced. Since many tumor-associated antigens or other epitopes or receptors which may be involved in diseases or which may play a role in the treatment of said diseases known. The method according to the present invention thus replaces the method known from the prior art approach by imprinting particle surfaces in the presence of such structures, e.g., antigens, epitopes or receptors, or changed or reduced segments thereof.

According to the present invention, it is possible to produce polymer matrices that simulate bispecific antibodies in that they are custom-made for specific epitopes of a transport cell and can be tethered to them before they are delivered into the bloodstream. A second molecularly imprinted binding site facilitates the recognition and binding to other cells, e.g., tumor cells. This can reduce the circulation time and increase the uptake rate at the target site if the transporting cells are selected to ensure that they react to the affected tissue.

Preferably, the binding cavities are for immune cells, in particular T-lymphocytes, monocytes or macrophages derived from monocytes, and for antigen-presenting cells. These binding cavities can also be for stem cells.

The polymer matrix preferably consists of a biological and/or natural polymer. The biocompatible polymer is preferably selected from the group comprising poly(meth)acrylic acid, poly(2-hydroxyethylmethacrylate), polyacrylamides, polylactides, polyglycosides, polyphosphazenes, polyorthoesters, polyanhydrides, poly(N-vinylpyrrolidone), poly(hydroxyalkanoates), polyurethanes, polysiloxanes, poly(ethylene oxide), poly(vinyl alcohol), poly(ethylene), poly(methylmethacrylate), poly(ethylene glycol), polyglycolides and copolymers, blends and mixtures of these. The natural polymers are preferably selected from the group comprising starch, cellulose, chitosan and copolymers, blends and mixtures of these. It is also possible to use inorganic materials, for example, silicon dioxide materials, in particular porous hollow particles thereof; titanium and alloys thereof; aluminum, calcium, titanium and cobalt oxides; hydroxyapatites, fluoroapatites and other calcium phosphates; aluminates, selenates and antimonates.

The specific binding cavities can preferably be produced by incorporation of specific epitopes, receptors or parts of these, which subsequently are once again removed from the polymer matrix. This removal can be effectuated, e.g., by using hydrolytic enzymes, by heating or by using suitable chemicals. It is similarly possible to produce the specific binding cavities by incorporating specific proteins, polysaccharides, fats and/or nucleic acids into the polymer matrix and by subsequently hydrolyzing them by means of lysosomal enzymes, which generally leads to a dissolution of the outside structure, which, as a rule, is the shell.

The preferred type of polymer matrix is a particle, in which case the particle surface comprises the binding cavities. Such particles preferably have a grain size in the range from 10-1000 nm.

According to another preferred embodiment, the polymer matrix contains a magnetic or magnetizable material, e.g., an iron-containing material such as magnetite, maghemite or hematite. Particles of this type make it possible to heat selective regions in the body, to which regions the particles bind due to the molecularly imprinted surface structure. The selective regions are heated by exciting the particles by means of radio waves or microwaves. Depending on the degree of the induced temperature increase, this increase can be used, e.g., to release active substances from temperature-sensitive particles or to heat the target tissue to kill the relevant cells. Magnetic nanoparticles of this type can be obtained either by using known synthesis methods or are enveloped with a biocompatible and biodegradable polymer, e.g., of lactic acid, urethane anhydrides, siloxane, vinyl alcohols, acrylamide, ethylene, ethyl-2-cyanoacrylate, gelatins, dextran or mixtures of the above. According to another embodiment of the manufacturing method, the magnetic material is incorporated into the polymer matrix during polymerization.

According to yet another preferred embodiment, the polymer matrix contains at least one active substance. This makes it possible to enhance the action, e.g., of T-cells at the site of action by releasing the active substance. In this manner, by loading the polymer matrix with cytostatics, a targeted release of active substance at the tumor site can be effectuated. Such polymer matrices that are loaded with the active substance and can have the form, e.g., of particles, can either be injected into the bloodstream or they can first be combined ex vivo with cells, e.g., T-lymphocytes or monocytes, before these complexes are injected into the bloodstream. As to loading the particles with the active substances, no restrictions apply; thus, both the inside and the surface of the particles can be loaded. In the former case, the imprinted outside surface is designed so as to become permeable to the drug or to dissolve, either after a certain length of time or mediated by the binding process to the target epitope as a result of a changed pH value in the target tissue or in certain compartments, or mediated by exocytosis of the acid cytolytic content of . . . T-lysosomes after binding to the cell to be treated via the T-cell receptor. The treatment of cancer is a classic example of such an application.

Generally, the active substance can be released by diffusion, by (biological) degradation of the substrate material and by swelling with subsequent diffusion or a combination of the above. The release of the active substance can be time-controlled, mediated by desorption, pH-dependent, mediated by lysosomal hydrolysis and/or by magnetic interaction. An extended-release action may be preferable.

In addition, polymer matrix systems that are able to change their size by swelling or shrinking may be made to release their active substances as a result of the following environmental parameters: temperature; ionic strength; charged compounds that are secreted by the target cells and that cause the system to swell due to the resulting electrostatic repulsion; and electron donor compounds which can enter into charge-transfer complexes with the systems.

The different release mechanisms possible will once again be briefly described below.

### 1) pH Dependent

The incorporation of the MIP-surrounded particles into the bloodstream leads to a tethering to T-leukocytes and to the transport to the target site. The reaction of the T-cell to the cancer cell leads to the excretion of lysosomes in association with a decrease in the pH value. This decrease causes the pH-sensitive microgels to excrete the drug.

When pH-sensitive protein units are used, the network is cleaved at these sites; with the other microgels, the network reversibly swells and decreases, which causes the solvent contained in the microgel to be excreted with the drug.

### 2) Lysosomal

With polymer units that were functionalized with enzyme-sensitive segments, lysosomal hydrolysis leads to cleavage of the polymer network.

### 3) Time-controlled

From simple core-shell particles, the active substance is released by the slow dissolution of the shell that contains the drug, and from particles in which the core contains the active substance, the active substance is released by the dissolution of the shell that acts as barrier.

4) pH-dependent (Enhanced by Chain Reaction)

After desorption of the particle/T-cell unit and subsequent renewed coupling to other epitopes of the cancer cell, the use of pH-sensitive particles leads to a further excretion of lysosomes. Thus, the pH value in the environment is gradually reduced, which finally leads to the complete dissolution of the particle.

### 5) Magnetic Interaction

An alternate magnetic field causes the particles to heat up and the polymer structure to be destroyed, which leads to the excretion of the drug. In the temperature-sensitive microgels, the polymer contracts, and the solvents contained in the cavities are squeezed out. In this case, the temperature threshold used is sufficiently high to avoid a premature release of the drug. The magnetic particles can be localized in the body by means of magnetic detection (SQUID) and make it possible to specifically start the reaction as soon as a sufficient accumulation in the tissue has occurred.

An applied magnetic field can furthermore lead to a change of the pores in the gel, and an electrical field can lead to a change in the charge of the membrane and to a migration of a charged active substance. In both cases, the effect is that the swelling behavior is changed, which causes the active substance to be released.

In addition, ultrasound can also lead to an increase in temperature and thus to a release of the active substance.

As to the method of binding the active substance to the polymer matrix, no limitations apply. The interactions preferably are based on physical and chemical adsorption to the surface of the polymer matrix. However, it is also possible for the active substance to be incorporated into the polymer matrix.

According to yet another embodiment of the polymer matrix according to the present invention, said polymer matrix has the form of a hydrogel. Hydrogels of this type preferably have a grain size in the range between 100 and 1000 nm. To produce such a hydrogel, a solution containing the relevant monomer units and a crosslinker, e.g., tetraethylene glycol dimethacrylate (EDGMA) or pentaerythritol tetraacrylate (PETEA), and a crosslinker which contains, e.g., functional groups, such as acrylamide units that can be bound to the amide nitrogen via aliphatic, aromatic or heteroaromatic spacers, are dissolved in a mixture of water and an organic solvent, such as ethanol. In the production of such hydrogels according to the present invention, it is also possible, by incorporating certain protein or polysaccharide chains, fats or nucleic acids into the polymer, to create functional segments which can be hydrolyzed by lysosomal enzymes and which cause the microgel to dissolve. Similarly, pH-sensitive protein units can be used. The radical initiators preferably used to produce the hydrogels are 4,4′-azobis-(4-cyanopentanoic acid) or 2,2′-azobis(isobutyronitrile). However, photolytic radical initiation by means of UV light is possible as well. According to another preferred embodiment, temperature-sensitive hydrogels are used, which can be produced, e.g., from poly-(N-alkylacrylamides). The temperature sensitivity of such hydrogels can be achieved by varying the crosslinker used and its concentration or by adding comonomers. The presence of magnetite or maghemite during the synthesis makes is possible to produce magnetic particles which can also react to external fields. As to the monomers, crosslinkers and solvents used for the polymer matrix, reference is hereby made to claims 20-22.

To produce specific cavities for the recognition of certain types of cells, e.g., T-lymphocytes or monocytes, the cells, their epitopes or regions of the epitopes are bound to a biopolymer surface, such as gelatin or dextran, that is located on a substrate. To this end, previously modified antibodies can be used by binding sulfhydryl groups or other functional groups to the biopolymer and the cell types mentioned above to the functional groups via avidin-biotin complexing by means of NeutrAvidin. By modifying the antibodies, e.g., by biotinylation, it is ensured that the antibody binds to the biopolymer layer in the orientation desired. The biopolymer is bound in the form of a thin layer to one side of a polymer die, as a result of which capillaries with a diameter of only few micrometers form. On the opposite side of the bound cells, characteristic markers or epitopes, such as the tumor marker EpCAM, are affixed. The targeted configuration of the two components on the particle can thus be achieved by means of the spatial configuration during the synthesis in that molecular imprinting takes place in the capillaries. The specific cavities form on the shell that forms in the presence of the drug-containing particles on opposites sides. By separating the polymer halves and washing them with solvents, the finished particles can be extracted.

According to another preferred embodiment, the surface epitope that produces the binding cavity in the polymer matrix binds to a sterically sufficiently demanding molecule, e.g., a polymer, or to a sufficiently large particle, e.g., gelatin particles, colloids or liposomes. This does not just create a very small number of cavities for the transport cell. The mutual hindrance of the epitope particles can also be produced by a high ionic charge of the epitope particles, which leads to repulsion. Similarly, even if each polymer particle has a plurality of cavities, targeted low loading with substrate particles can be achieved by a low concentration of substrates during the subsequent loading if the number of cavities is kept low during the polymerization of the polymer matrix by means of the methods mentioned.

When magnetically active colloids are used as epitope substrates, these can be made to move to one side of the polymerization vessel or polymerization channel by the specific application of a magnetic field, i.e., using the flow method. During the simultaneous polymerization of the polymer matrix with the second epitope of the target molecule that is tethered to the opposite side, the cavities form correspondingly on the side facing away from the target molecule.

If the method above is used without the presence of the polymer die, the configuration of the cavities will take place nonspecifically. In this case, the antibodies of the T-cells are bound to gelatin particles of sizes larger than 500 nm in order to prevent an imprinting of the antibody-carrying particles.

According to another preferred embodiment, nanoscale molecularly imprinted polymers are produced in the first step, which polymers have short oligopeptide subsegments as freely mobile or immobilized templates which consist of the amino acids of the proteins of the epitope selected, which amino acids are specific to the N- or C-terminal ends. These peptide-imprinted polymers, which can recognize the N- or C-terminal oligopeptides and thus the target epitope with high specificity and affinity, subsequently serve, in a second step, as a template for a stable prepolymerization complex with suitable functional monomers. In a third step, these are subsequently polymerized to produce more or less highly crosslinked chains (as an alternative, the functional monomers are already tethered into chains) which are held together by the noncovalent interactions between the functional monomers incorporated into the polymer and the peptide-imprinted polymer templates and thus can serve as substrates for active substances. When the peptide-imprinted polymers are bound to the target epitope, the polymer structure becomes loosened, which leads to a release of the active substance(s). The system described can, in addition, also be coupled to an MIP that is specific to immune cell epitopes.

Depending on the production method, the polymer matrix according to the present invention can take a number of different forms. These include:
Production of polymer monoliths and subsequent fragmentation
Grafting the imprinted polymer on preformed particles
Production of polymer beads from suspension, emulsion or dispersion polymerization
Polymer particles that are bound to thin layers or polymer membranes
Polymer membranes
Surface-imprinted polymer phases: The formed complexes of the template molecules with the functional monomers bind to activated surfaces, such as silicon or glass surfaces, and after washing, lead to defined imprinted structures.

To illustrate the potential uses, examples of a few useful applications follow. These include, for example, the target-specific detection, the covalent or noncovalent binding to the target and the controlled release of active substances in target tissues, which is a very important aspect of modem treatments since it makes it possible to reduce the concentration of the active substances drastically. One example is the use in cancer therapy in which the inhibition of receptors, e.g., the epidermal growth factor receptor (EGFR), is an important goal in order to suppress signal transmission. In contrast to the new systems according to the present invention, the prior-art monoclonal antibodies have the disadvantage that they are considerably more unstable, that they are immunogenic and that their production requires a higher degree of technical complexity. An additional advantage of the polymer matrix according to the present invention over the monoclonal antibodies is that it can be produced considerably less expensively.

Another application is the use in diagnostics by labeling the particles with fluorescing dyes or contrast agents. Molecular imprinting of the surface allows an interaction with specific target cells, e.g., directly in the body or in a blood sample.

In addition, the polymer matrix according to the present invention can also be combined to isolate the cells. The use of particles with a magnetic core and an outer molecularly imprinted surface layer thus makes it possible to isolate the bound cells in the magnetic field.

The subject matter of the present invention will be explained in greater detail using the following example, without however limiting the invention to the special embodiment presented here.

### EXAMPLE 1

Following the specification by Chen et al. (Jian-Feng Chen, Hao-Min Ding, Jie-Xin Wang, and Lei Shao, Biomaterials 25 (2004), pp. 723-727), hollow silica nanoparticles are produced as the substrate material for the active substance used and its controlled release, except that the sodium silicate component (Na₂SiO₃·9 H₂O) is added dropwise to the calcium carbonate suspension (grain size 90 nm) over a longer period of time (5-10 days) by means of a peristaltic pump. Subsequently, following the specification by Sellegren et al. (Bärbel Rückert, Andrew J. Hall and Börje Sellergren, J. Mater. Chem. 12 (2002), pp. 2275-2280), the p-(chloromethyl)phenyl groups can be introduced into the hollow silica nanoparticles thus produced and be coupled to the diethyl dithiocarbamate groups as radical initiators. The carbamate-derived sites now serve as the starting points for the UV-controlled grafting of molecularly imprinted poly(methacrylic acid/coethylene glycol dimethacrylate) copolymers; the templates used are oligopeptides which are contained in the N- or C-terminal sequences of the relevant target epitopes and which are specific to the epitopes selected.

1. A polymer matrix for the specific tethering of surface epitopes or receptors of a cell, said polymer matrix comprising molecularly imprinted surface epitopes or receptors and binding cavities that are specific to these surface epitopes or receptors.

2. The polymer matrix of claim 1, wherein the polymer matrix has molecularly imprinted binding cavities for at least one immune cell, for antigen-presenting cells, for tumor cells, or for at least one stem cell.

3. The polymer matrix of claim 2, wherein the polymer matrix is produced from a biocompatible and/or natural polymer.

4. The polymer matrix of claim 3, wherein
the biocompatible polymer is selected from the group consisting of poly(meth)acrylic acid, poly(2-hydroxyethylmethacrylate), polyacrylamide, polylactide, polyglycoside, polyphosphazene, polyorthoester, polyanhydride, poly(N-vinylpyrrolidone), poly(hydroxyalkanoate), polyurethane, polysiloxane, poly(ethylene oxide), poly(vinyl alcohol), poly(ethylene), poly(methylmethacrylate), poly(ethylene glycol), polyglycolide, and a copolymer, blend, or mixture thereof, and wherein
the natural polymer is selected from the group consisting of starch, cellulose, chitosan, and a copolymer, blend, or mixture thereof.

5. The polymer matrix of claim 1, wherein the specific binding cavities are produced by the incorporation of specific epitopes, receptors or parts thereof and are subsequently once again removed from the polymer matrix.

6. The polymer matrix of claim 1, wherein the specific binding cavities are produced by the incorporation of specific proteins, polysaccharides, fats and/or nucleic acids, with subsequent hydrolysis by lysosomal enzymes.

7. The polymer matrix of claim 1, wherein the polymer matrix contains a magnetic or magnetizable material.

8. The polymer matrix of claim 1, wherein the polymer matrix comprises at least one active substance.

9. The polymer matrix of claim 1, wherein the release of the active substance is in a time-controlled manner, mediated by desorption, pH-dependent, mediated by lysosomal hydrolysis, initiated by compounds excreted by the cells, initiated by electron donor compounds and/or by magnetic interaction.

10. The polymer matrix of claim 8, wherein the active substance can be released over an extended period of time.

11. The polymer matrix of claim 8, wherein the at least one active substance is bound to the surface of the polymer matrix by way of physical or chemical adsorption and/or is incorporated into the polymer matrix.

12. The polymer matrix of claim 1, wherein the polymer matrix comprises at least one contrast agent and/or at least one dye.

13. The polymer matrix of claim 1, wherein the polymer matrix has the form of a particle and the particle surface has binding cavities.

14. The polymer matrix of claim 13, wherein the particle has a grain size in the range between 10 and 1000 nm.

15. The polymer matrix of claim 1, wherein the polymer matrix has the form of a hydrogel.

16. The polymer matrix of claim 15, wherein the hydrogel comprises at least one active substance, and the release of the active substance is mediated by the pH sensitivity of the hydrogel.

17. The polymer matrix of claim 15, wherein the hydrogel comprises at least one active substance, and the release of the active substance is mediated by the temperature sensitivity of the hydrogel.

18. A method of producing a polymer matrix with binding cavities for surface epitopes or receptors of cells, wherein a polymer matrix is produced by crosslinkage and that specific proteins, polysaccharides, fats and/or nucleic acids are bound to the surface of the matrix by way of physical and/or chemical adsorption or are incorporated into the matrix.

19. The method of claim 18, wherein the polymer matrix is produced from at least one monomer and at least one crosslinker in the presence of a radical initiator in a solvent.

20. The method of claim 19, wherein the polymer matrix is produced from monomers selected from the group consisting of carboxylic acid and their amides, sulfonic acid, a heteroaromatic base, an aliphatic or aromatic vinyl compound with a chelating group and a mixture thereof.

21. The method of claim 19, wherein the at least one crosslinker is selected from the group consisting of a divinylbenzene crosslinker, a (meth)acrylic acid crosslinker, a tri- or tetrafunctional crosslinker, an acrylamide crosslinker, and a mixture thereof.

22. The method of claim 19, wherein the solvents used in the polymerization are aliphatic or alicyclic hydrocarbons, aromatic hydrocarbons, halogenated hydrocarbons, alcohols, ether, acetronitrile, tetrahydrofuran, ethyl acetate, acetone, dimethylformamide, dioxane, dimethylsulfoxide, a mixture thereof, or a mixture thereof optionally mixed with water.

23. The method of claim 19, wherein the initiator used for the polymerization is a radical initiator, and/or UV radiation.

24. A method for inhibiting a receptor for therapeutic purposes comprising contacting the polymer matrix of claim 1 with a receptor on a cell, wherein the receptor is inhibited.

25. The method of claim 24, wherein the method is for the target-oriented delivery and the release of active substances.

26. A method for diagnosing a disease pathogen comprising administering the polymer matrix of claim 12 to a subject comprising a receptor on a cell.

27. A method for isolating cells in a magnetic field comprising contacting at least one cell with the polymer matrix of claim 7, applying a magnetic field to the cell, and isolating the cells.
